(19) United States
(12) Patent Application Publication
(10) Pub. No.: US 2016/0132226 A1
(43) Pub. Date: May 12, 2016

(54) BLOOD PURIFICATION DEVICE FEEDBACK METHOD

(71) Applicant: B. BRAUN AVITUM AG, MELSUNGEN (DE)

(72) Inventors: MÁTÉ BOCZ, BUDAPEST (HU); ZSÓFIA BÉKY, BUDAPEST (HU)

(21) Appl. No.: 14/926,782

(22) Filed: Oct. 29, 2015

(30) Foreign Application Priority Data
Nov. 10, 2014 (EP) 14192498.5

(51) Int. Cl. G06F 3/0484; A61M 1/16; A61M 1/36

(52) U.S. Cl. CPC G06F 3/04847; A61M 1/3607; A61M 1/1603

(57) ABSTRACT

A method for providing feedback on an expected and/or required user interaction in a blood purification device is disclosed. The method includes monitoring, at a current position in an operational flow of the blood purification device, whether a user interaction is expected and/or required in a subsequent step in the operational flow. If it is determined in the monitoring step that a user interaction is required and/or expected, the kind of required and/or expected user interaction is determined. A user interaction feedback indicating the determined kind of user interaction required and/or expected in the at least one subsequent step is then proactively activated at an active element corresponding to the user interaction in a graphical user interface.

### CROSS REFERENCE TO RELATED APPLICATIONS

This application claims priority to European application EP 14192498.5 filed Nov. 10, 2014, the contents of such application being incorporated by reference herein.

### FIELD OF THE INVENTION

The invention relates generally to blood purification and renal replacement therapy. More specifically, the invention relates to monitoring device operation during blood purification and renal replacement therapy, and to generate user feedback based on an analysis of the monitored device operation.

### BACKGROUND OF THE INVENTION

Blood purification devices are complex systems and as such combinations of hardware and software components. Operators of such machines are often not aware of what they are required and/or expected to do next, whether or not the machine needs interaction, and if so, how to interact.

Among known methods and systems, a device may be designed to include a number of functions for controlling parameter values, i.e. parameter control functions, to enable a user to quickly and easily control variable parameter values using a graphical user interface and to enable the user to obtain the desired precision of control of such variables. One such system provides e.g. four control functions: a data entry function, single step function, a scroll function, and a translation function. The user can select the particular control function used to control a parameter value in light of the particular amount or type of control that needs to be accomplished.

Another known medical apparatus comprises a user interface for setting parameters and includes a screen for visualizing values of said parameters, a main control unit connected to the interface, a first memory and a video memory both connected to the main control unit for storing data corresponding to images on screen. The main control unit allows setting of a new value for a parameter, displays the new value on a screen region, stores the new value in the first memory, captures from the video memory data representative of said screen region, and verifies from said representative data if the displayed value corresponds to the value in the first memory.

A further known arrangement includes an enclosure, a medical therapy machine component located inside the enclosure, a video monitor supported by the enclosure and displaying at least one of information and indicia relating to the medical therapy, an ambient light sensor positioned with respect to the enclosure so as to be able to sense a level of ambient light impinging the enclosure, and a logic implementer configured to control a level of backlight brightness for the video monitor based on the level of ambient light sensed by the sensor.

While, accordingly, known medical devices often employ icons, sound, animations, and the like on a graphical user interface to give feedback to the operator on the status of the device elements, the graphical user interfaces of device for renal replacement therapies can be quite complex and crowded since the devices themselves are complicated. The graphical user interface displays important information and provides means for the operator to trigger events. Having too many elements on screen at once can, however, result in low usability of the device. It is therefore important that the operator can instantly find all the information he is looking for, and is supported by the device on what he needs to do.

Arrangements of the exemplified known kind do not motivate any suitable concept in a blood purification device toward providing enough feedback on whether a user interaction is required, and which user interface element is concerned, with a view to improved user confidence and usability.

### SUMMARY OF THE INVENTION

In view of the above, an object of the invention resides in providing a renal replacement therapy and/or blood purification device and a method therefor that are capable of providing sufficient guidance to an operator regarding user action the device is expecting from him.

In addition, the invention shall provide a method for drawing a user's attention to specific elements of the user interface and helping him even on a crowded screen.

According to aspects of the invention, this object is accomplished by a method for providing feedback in a blood purification device and by a blood purification device arranged to carry out the method asset forth in the independent claims. Advantageous further developments of the invention are subject of the accompanying dependent claims.

According to a basic concept underlying the invention, a graphical user interface consists of various elements, such as buttons, icons, boxes, values, texts etc. Such user interface elements can be active, meaning that the operator can interact with it by e.g. touching it or performing a gesture. This action triggers an event within the device. The idea of the invention is to add either a static or animated feature (e.g. coloring, blinking) to the active user interface element in order to catch the users' attention and direct it to the element, to suggest that an action is expected from the user, and/or to suggest the need for interaction with the element. Within this concept, user interface elements proactively indicate that a user interaction is required. Indication can be in form of any graphical highlighting or change, such as dynamically changing color, shape, opacity, etc.

Advantageous effects of the invention include improved usability by directing the user's attention to the user interface element that requires user action. This enhances perception, reduces cognitive load, thus speeding up user interaction and boosting user confidence. In addition, the invention can in general provide help and hints to the user on expected user actions, and an operator can be aware of which parameters are required and need to be set before proceeding to next user actions, aware that one of the buttons needs to be interacted with. Also, these advantageous effects increase the user's confidence while he uses the device, and improves the quality of involved human-machine interaction, which in turn enhances device usability.

Thus, according to an aspect of the invention, the object is accomplished by a method for providing feedback on an expected and/or required user interaction in a blood purification device, comprising the steps of monitoring, at a current position in an operational flow of the blood purification device, whether a user interaction is expected and/or required in at least one subsequent step in the operational flow; if it is determined in said monitoring step that a user interaction is required and/or expected, determining the kind of required and/or expected user interaction; and proactively activating a user interaction feedback indicating the determined kind of user interaction required and/or expected in said at least one subsequent step at an active element corresponding to the user interaction in a graphical user interface.

Preferably, there is provided also a step of selecting a static or an animated user interaction feedback in accordance with the kind of user interaction determined in said required user interaction determining step. In a graphical user interface, soft buttons as active elements can be arbitrarily designed and assigned more than one function in their vicinity. Accordingly, an active element can have assigned more than one user feedback indication. A static user interaction feedback can then, for example mean “press this button”, and an animated user interaction at the same button can then indicate “press this button again”, and the like.

Therefore further preferably, said proactively activating step comprises activating said user interaction feedback as a fixedly colored, static frame, a gradient of colors on a static frame, a fixedly colored and repeatedly appearing and disappearing frame, a changing color static frame, a blinking frame with variable blinking speed, and/or a fixedly colored, alternately growing and shrinking frame.

Still further preferably, there is provided a step of setting at least one control parameter for said user interaction feedback, said parameters controlling an appearance of the user interaction feedback at said active element.

In this respect, it is advantageous when said at least one control parameter includes a color parameter defining the color in which an active element surrounding frame, as said user interaction feedback appears, and/or or color-pairs a gradient uses as a starting and ending gradient color, a speed parameter defining the speed of a user interaction feedback animation, a border weight parameter defining a thickness of the active element surrounding frame, and/or an enable parameter controlling a visibility of the user interaction feedback.

Also preferably, there is provided a step of changing the position, size and/or type of an animation of the user interaction feedback at the active element.

Next preferably, spare user interaction feedback variants not assigned to a determinable user interaction are kept available for use in an escalating step enhancing a user interaction feedback set for a determined user action beyond normal in case of occurrence of an exceptional device state. In practice, unforeseen exceptions such as emergency halt conditions, defective components, unstable controls and immediate alert requirements not stemming from the currently triggered event and/or its states may occur. Being able to overdrive regular control and feedback indication is advantageous in such cases to help catching the user's immediate attention.

In addition preferably, there is provided a step of resetting the user interaction feedback at the active element upon detection that the determined user interaction of a completed has triggered an event in the device and/or has been carried out. Thereby, any feedback representation is also halted, and the user can easily get confirmation of his successful interaction and, if a next feedback is given at the same or another active element, know what he is expected to do next.

According to a further aspect of the invention, an object is also accomplished by a blood purification device comprising components assigned to a monitorable operational flow and a graphical user interface including one or more active elements arranged to trigger one or more events in the device and to present user interaction feedback on user interaction determined and/or expected based on a monitoring of said monitorable operational flow, the blood purification device being arranged to carry out the method according to one of the preceding steps. Advantageously, the method can be operated on any blood purification or renal therapy device or machine equipped with supporting hardware and arranged to carry out the method.

### BRIEF DESCRIPTION OF THE DRAWINGS

The invention is best understood from the following detailed description when read in connection with the accompanying drawings. Included in the drawings are the following figures:

FIG. 1 schematically shows a flow chart of an operation flow in a blood purification device providing user interaction feedback to an active element in a graphical user interface of the device according to a preferred embodiment; and

FIG. 2 schematically exemplifies feedback presentations to a user according to the control carried out in the embodiment.

### DETAILED DESCRIPTION OF THE PREFERRED EMBODIMENTS

Generally referring to the preferred embodiment, the configuration thereof basically relies on supporting hardware provided in any blood purification device/machine or blood purification related device benefiting from the embodied blood purification device feedback method. Such hardware can, therefore, in particular comprise sensors, detectors, pumps, filters and various other process and measurement means of which a state or status can be queried, polled and/or sampled, or that deliver a certain value during device operation, and on suitable means arranged to process such queried status and/or values and generate an output based on the processing result.

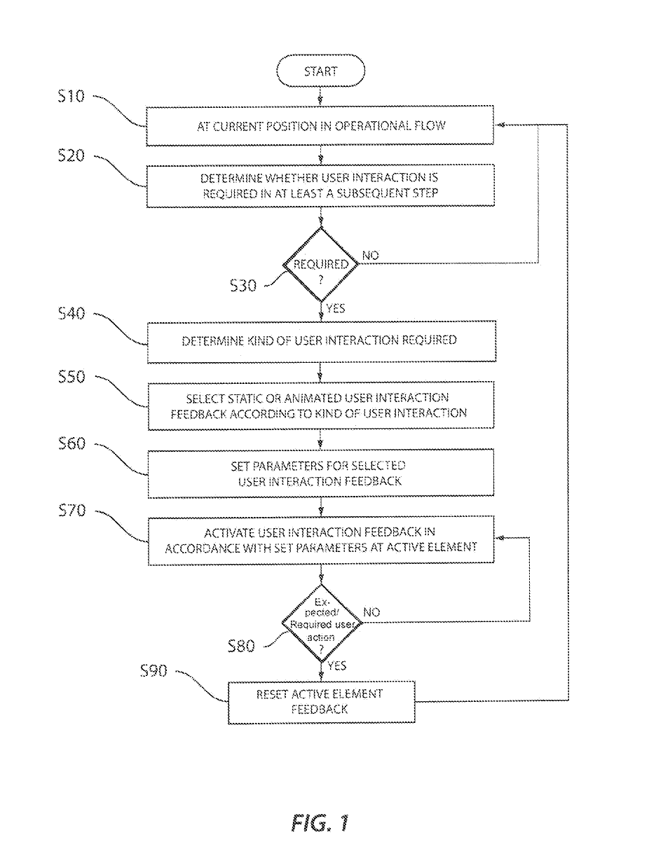

FIG. 1 schematically shows a flow chart of an operation flow in a blood purification device providing user interaction feedback to an active element in a graphical user interface of the device according to a preferred embodiment.

A processing in the underlying device monitors the operational flow thereof and branches into the flow of the present embodiment at a step S10, where a current position in the operational flow is sort of pinpointed or marked for the purpose of the present embodiment. The process then proceeds to a step S20.

In step S20, it is determined whether a user interaction is required at at least one step of the operational flow subsequent to the current step. In other words, the control proactively looks ahead, or further “down” the operational flow it is working, as to a user will be required to take any action via the graphical user interface of the device later on.

This looking ahead can for example include a predetermined number of subsequent steps and more specifically be configured to leave sufficient processing time to activate the user interaction feedback at the corresponding active element in time and without generating any waiting states, delay and the like. I.e. a user interaction feedback at the active element can be given before the device or operational flow actually requires and/or expects the user interaction to be carried out

If no user interaction is required according to a check to this effect in a next step S30, the process returns to step S10 in order to keep track of the current position in the operational flow and to keep looking ahead the configured step range as to whether a user interaction requirement is involved in any of later operational flow positions. In other words, steps S10 to S30 are looped until a user interaction requirement is detected “down the road”.

If a user interaction is required as the result of the checking in step S30, the process proceeds to a step S40, in which a kind, type, group, category etc. of the required user interaction is determined. The kind of user interaction can be used to switch and/or select between different user interaction feedback at the active element, i.e. to select a certain feedback for a certain kind of user interaction, and to select a different feedback for a different kind of user interaction. The number of distinguishable kinds of user interactions is not particularly limited but can generally correspond to the number of representable feedback indications at one or the active element(s), and it is understood that it also be only one in a more simple case. After determining the kind of user action required, the process proceeds to a step S50.

In step S50, a static or animated user interaction feedback is selected according to the kind of user interaction determined in step S40. This selecting step provides for the setting of sort of a basic type of user interaction feedback. The process then proceeds to a next step S60.

In step S60, parameters configuring the basic type of user interaction feedback selected in step S50 into a desired form.

In the present embodiment, a graphic (either static or animated) is placed on or around the button. The graphic follows the outline of the button with the sides being colored and/or animated and the middle being transparent so that the button can be seen. When animated, the animation can have various forms, and for example be represented by a fixedly colored, static frame, a gradient of colors (e.g. two colors) on a static frame, a fixedly colored, repeatedly appearing and disappearing frame, a changing color static frame, a blinking frame in which the blinking speed changes, a fixedly colored, growing and shrinking frame, and the like.

The animation as such can be used for e.g. marking parameters that need to be set (required parameters) with a fixedly colored, static frame around them, and/or marking buttons that need to be pressed as an expected next user action by a fixedly colored, repeatedly blinking frame. Indication as such can be in the form of arbitrary graphical highlighting or modification, such as dynamically changing color, shape, opacity, etc. Further modifications can change the position or size of a user interface element, or introduce other animated features (e.g. spinning).

Accordingly, in step S60, as example parameters and without limitation thereto, a color parameter defines the color in which a frame appears or what color-pairs the gradient uses as a starting and ending gradient color. A speed parameter can define the speed of an animation. When set to 0, the frame is static, and when set to other than 0, the frame is either rotating the angle of gradient or repeatedly changes an opacity value of the color from 0 to 100 and back in order to provide a blinking effect. A border weight parameter defines the number of pixels of the thickness of the frame. An enable parameter is used to control the visibility of the animation. When the enable parameter is set to 1, the animation is active and visible, and when it is set to 0, the animation is hidden.

In a subsequent step S70, the current user interaction feedback is activated at the active element in accordance with the previously set definition thereof.

After the currently applicable user interaction feedback has been set to the active element in step S70, the process proceeds to a step S80. In step S80, it is confirmed whether or not the required and/or expected user interaction has taken place, or been carried out by the user.

If it is found in step S80 that the user interaction is not completed, or has not taken place, the process returns to step S70, i.e. steps S70 and S80 are looped in order to keep the user interaction feedback at the active element alive in its current representation until the user takes action as required and/or expected. Once done, the process proceeds to a step S90.

In step S90, the user task having been carried out as required and/or expected is taken as finalized, and accordingly the user interaction feedback at the active element is reset to a predetermined non-feedback state easily recognizable as such by the user. Resetting the user interaction feedback at the active element may, for example, include turning off a lit state, halting an animation, changing a color to standard or neutral, and the like, and in connection therewith, process variables such as the determined kind of user interaction feedback, the selected type of basic representation, and any parameters varying the basic representation may also be returned to an initial state from where to suitably proceed from in a next process run.

To trigger such a next run, the process in the present embodiment is designed to jump back to step S10 from step S90, i.e. is configured as an endless loop as long as the blood purification device is operative. In this way, permanent monitoring and user interaction feedback as long as the device is operative, is achieved. It is, however, understood that the present invention is not limited to such permanent looping, and that a different process exit strategy can be used.

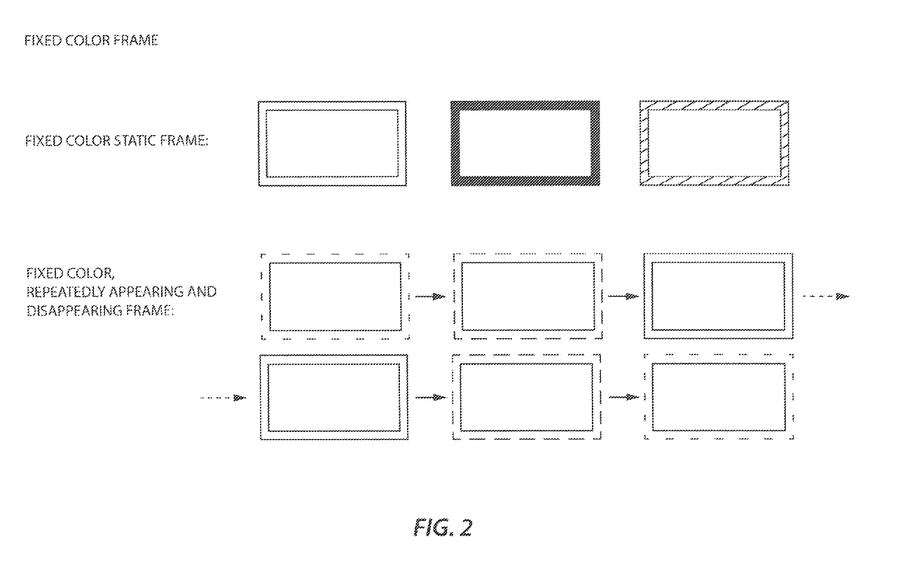

Turning to FIG. 2, feedback presentations to a user as examples of the user interaction feedback according to the control carried out in the embodiment are schematically illustrated based on a rectangle as basic form suitable for an exemplary rectangular active element or button. It is, however, understood that various representations other than a rectangle are possible.

To this effect, the upper half of FIG. 2 depicts a static frame of fixed color placed around the button as the active element. It is noted that the color is not limited to a specific one. The lower half of FIG. 2 shows a repeatedly appearing and disappearing frame of fixed color at a same button (following the arrows indicates the changes at a same button from not present/disappeared in dashed line with shorter dashed, to partially present in dashed line with longer dashes, to present/appeared in full line, and vice versa. Of course, there may be less or more intermediate stages than are shown in FIG. 2.

Insofar, along the preferred embodiment and teaching disclosed herein, the invention provides solutions for problems arising from an operator having no or little guidance on whether any user action on the graphical user interface is expected by the device, an operator having no or little guidance on which graphical user interface element needs to be interacted with, and an operator having no or little guidance on how to interact with the user interface element.

While the invention has been described with reference to a preferred embodiment and the accompanying drawings, it is understood that the present invention is not in any way limited to particular details disclosed with respect to this preferred embodiment, and that any modification readily apparent to the skilled person based on the here presented teaching is deemed to be within the scope of protection as defined by the appended claims.

1-9. (canceled)

10. A method for providing feedback on at least one of an expected or required user interaction in a blood purification device, comprising the steps of:
monitoring, at a current position in an operational flow of the blood purification device, whether a user interaction is at least one of expected or required in at least one subsequent step in the operational flow;
if it is determined in said monitoring step that the user interaction is at least one of required or expected, determining a kind of user interaction for the at least one required or expected user interaction; and
proactively activating a user interaction feedback indicating the determined kind of user interaction for the at least one required or expected user interaction in said at least one subsequent step at an active element corresponding to the at least one required or expected user interaction in a graphical user interface.

11. The method according to claim 10, further comprising the step of:
selecting a static or an animated user interaction feedback as the user interaction feedback based on the determined kind of user interaction.

12. The method according to claim 10, wherein said proactively activating step comprises activating said user interaction feedback as at least one of a fixedly colored, static frame, a gradient of colors on a static frame, a fixedly colored and repeatedly appearing and disappearing frame, a changing color static frame, a blinking frame with variable blinking speed, or a fixedly colored, alternately growing and shrinking frame.

13. The method according to claim 10, further comprising the step of:
setting at least one control parameter for said user interaction feedback, said at least one control parameter controlling an appearance of the user interaction feedback at said active element.

14. The method according to claim 13, wherein said at least one control parameter includes at least one of a color parameter defining at least one color in which an active element surrounding frame as said user interaction feedback appears or a gradient uses as a starting and ending gradient color, a speed parameter defining the speed of a user interaction feedback animation, a border weight parameter defining a thickness of the active element surrounding frame, or an enable parameter controlling a visibility of the user interaction feedback.

15. The method according to claims 10, further comprising the step of:
changing position, size and/or type of an animation of the user interaction feedback at the active element.

16. The method according to claim 10, further comprising the step of:
enhancing the user interaction feedback with a spare user interaction feedback variant that does not correspond to the at least one required or expected user interaction in response to an exceptional device state.

17. The method according to claim 10, further comprising the step of:
resetting the user interaction feedback at the active element upon completion of the at least one required or expected user interaction, upon triggering an event corresponding to the at least one required or expected user interaction, or upon completion of the event.

18. A blood purification device comprising components assigned to a monitorable operational flow and a graphical user interface including one or more active elements arranged to trigger one or more events in the device and to present user interaction feedback based on at least one of user interaction determined or user interaction expected based on a monitoring of said monitorable operational flow, the blood purification device being arranged to carry out the method according to claim 10.
